(19) United States
(12) Patent Application Publication
(10) Pub. No.: US 2013/0336889 A1
(43) Pub. Date: Dec. 19, 2013

(54) NANOPARTICLE AND METHOD FOR DETECTING OR TREATING A TUMOR USING THE SAME

(71) Applicants: Ming-Jium Shieh, Taipei (TW); Cheng-Liang Peng, Taipei (TW); Tsai-Yueh Luo, Taipei (TW)

(72) Inventors: Ming-Jium Shieh, Taipei (TW); Cheng-Liang Peng, Taipei (TW); Tsai-Yueh Luo, Taipei (TW)

(73) Assignee: NATIONAL TAIWAN UNIVERSITY, Taipei (TW)

(21) Appl. No.: 13/523,093

(22) Filed: Jun. 14, 2012

(51) Int. Cl. A61K 49/00; A61P 35/00; C08G 63/91; A61K 51/06; A61K 49/04; B82Y 5/00

(52) U.S. Cl. 424/1.89; 424/9.1; 424/1.65; 424/1.85; 424/9.6; 424/9.4; 525/54.2; 525/450; 977/773; 977/788; 977/915; 977/927; 977/928

(57) ABSTRACT

A nanoparticle for detecting or treating a tumor is provided. The nanoparticle includes a plurality of polymer backbones and at least one first detectable substance, of which each of the polymer backbones includes a hydrophobic region, a hydrophilic region and a chelating region, and the first detectable substance is bound to the chelating region of the polymer backbone. The hydrophobic regions of the polymer backbones form a core block, and the hydrophilic regions of the polymer backbones form a shell block surrounding the core block. A method for detecting or treating a tumor using the nanoparticle is also provided.

### BACKGROUND OF THE INVENTION

1. Field of the Invention

The present application relates to a nanoparticle, and more particularly relates to a nanoparticle for detection and treatment of a tumor.

2. Description of Related Art

Photothermal therapy (PTT) destroys cancer cells by generating heat within a tumor by absorbing specific light sources. A major challenge of thermal therapies is to selectively injure the targeted tissue without damaging the normal tissue Minimally invasive cancer treatments are currently being investigated, such as radiofrequency ablation, magnetic thermal ablation, focused ultrasound ablation and laser-based PTT. The effectiveness of such treatments is limited by nonspecific heating of targeted tissue, which often injures healthy tissue.

Exogenous chromophores are known to increase heat generation within targets by increasing the light sensitivity of targeted tissue. Therefore, exogenous chromophores that strongly absorb light in the near-infrared (NIR) region (650-900 nm) have been widely studied because they produce localized cytotoxic heat upon NIR irradiation. Since the tissue absorption of NIR light is minimal, it can penetrate deep into the tissue.

Polymethine cyanine dyes such as indocyanine green (ICG) are suitable contrast agents for clinical and experimental NIR imaging. ICG also exhibits unique optical properties due to its strong absorption at NIR wavelengths, which causes photothermal effects that can trigger thermal injury and cell death both in vitro and in vivo.

For hydrophobic dyes, not like ICG, polymeric nanoparticles have shown great promise in drug delivery due to their good biocompatibility, high stability both in vitro and in vivo, and successful encapsulation of various poorly soluble agents. An additional benefit of nanosized carriers is that they slowly accumulate in pathological sites, including tumors, through the enhanced permeability and retention (EPR) effect, which is known as a passive targeting. Many tumor tissues are supplied by a leaky neovasculature with an incomplete endothelial barrier and poor lymphatic drainage. The EPR phenomenon provides an opportunity for nanosized carriers to reach their target site.

However, a multifunctional nanoparticle for optical and nuclear imaging as well as for PTT is not yet available.

### SUMMARY OF THE INVENTION

A nanoparticle having biodegradability and biocompatibility comprising a plurality of polymer backbones and at least one first detectable substance for detecting or treating a tumor is provided, wherein each of the plurality of polymer backbones includes a hydrophobic region, a hydrophilic region, and a chelating region, and the first detectable substance is bound to the chelating region of the polymer backbone. In one embodiment, the hydrophobic regions of the polymer backbones form a core block, and the hydrophilic regions of the polymer backbones form a shell block surrounding the core block.

In one embodiment, the hydrophilic region comprises at least one of polyethylene glycol and polypropylene glycol, and the hydrophobic region comprises at least one of polycaprolactone, polybutyrolactone and polyvalerolactone. In one embodiment, the polymer backbones form a micelle. In one embodiment, the nanoparticle further comprises crosslinkages between the polymer backbones.

In one embodiment, the first detectable substance is a radionuclide selected from the group consisting of Fluorine-18, Copper-64, Technetium-99m, Indium-111, Iodine-123, Iodine-131, Holmium-166, Rhenium-188, Gold-198, and a combination thereof, wherein Rhenium-188 is used for detecting or treating liver cancer, colon cancer, breast cancer, lung cancer and a combination thereof, and Iodine-131 is used for detecting or treating liver cancer, thyroid cancer, neuroblastoma, glioblastoma, lymphoma, myeloma, and a combination thereof.

In one embodiment, the nanoparticle further comprises a second detectable substance bound to the hydrophobic region or the hydrophilic region of the polymer backbone, wherein the second detectable substance is a visible or near infrared detectable substance selected from the group consisting of fluorescein, fluorescein isothiocyanate (FITC), rhodamine, Texas Red, cyanine dye, cy3, cy5, cy5.5, cy7, cy7.5, Alexa fluor dye, heptamethycyanine, indocyanine green (ICG), IR-780, IR-783, ADS7800H, NIR-797 isothiocynate, and a combination thereof.

In one embodiment, the polymer backbones bound with a first detectable substance or a second detectable substance is between 1% wt and 100% wt, preferably between 5% wt and 100% wt, and more preferably between 10% wt and 100% wt. In one embodiment, the molecular weight of the nanoparticle is between 200 and 30000.

In one embodiment, the nanoparticle further comprises an anti-cancer drug, wherein the anti-cancer drug is selected from the group consisting of 7-ethyl-10-hydroxycamptothecin (SN-38), camptothecin (CPT), paclitaxel, doxorubin, 17-(Allylamino)-17-demethoxygeldanamycin (17-AAG), celecoxib, capecitabine, docetaxel, epothilone B, Erlotinib, Etoposide, GDC-0941, Gefitinib, Geldanamycin, Imatinib, Intedanib, lapatinib, Neratinib, NVP-AUY922, NVP-BEZ235, Panobinostat, Pazopanib, Ruxolitinib, Saracatinib, Selumetinib, Sorafenib, Sunitinib, Tandutinib, Temsirolimus, Tipifarnib, Tivozanib, Topotecan, Tozasertib, Vandetanib, Vatalanib, Vemurafenib, Vinorelbine, Vismodegib, Vorinostat, ZSTK474 and a combination thereof.

In another embodiment, a method for detecting or treating a tumor is provided. The method comprises administering a nanoparticle to a subject in need thereof, wherein the nanoparticle comprises a plurality of polymer backbones, each including a hydrophobic region, a hydrophilic region and a chelating region, and at least one first detectable substance bound to the chelating region of the polymer backbone. The hydrophobic regions of the polymer backbones form a core block, and the hydrophilic regions of the polymer backbones form a shell block surrounding the core block. In one embodiment, the nanoparticle further comprises a second detectable substance bound to the hydrophobic region or the hydrophilic region of the polymer backbone.

In one embodiment, the method further comprises detecting the first or second detectable substance by single-photon emission computed tomography (SPECT), positron emission tomography (PET), a radiation image system or a fluorescent image system.

In another embodiment, a composition for detecting and treating a tumor, comprising the nanoparticle and a pharmaceutical acceptable excipient thereof is provided.

### BRIEF DESCRIPTION OF THE DRAWINGS

FIG. 1 shows a schematic diagram illustrating one embodiment of the fabrication of the nanoparticle of the present invention.

FIG. 2 shows ¹H NMR spectra of (A) mPEG₅ₖ-PCL₁₀ₖ copolymer in CDCl₃. The characteristic resonances of both PCL (δM^{e}=1.38 ppm, δ^{d}=1.65 ppm, δH^{c}=2.28 ppm, δH^{c}=4.07 ppm) and PEG (δH^{a}=3.39 ppm and δH^{b}=3.65 ppm) were observed, suggesting the coexistence of two blocks. (B) Fmoc-PEG₅ₖ-PCL₁₀ₖ copolymer in CDCl₃. The ¹H NMR spectrum of Fmoc-NH-PEG-b-PCL exhibited distinct resonance signals of Fmoc moieties at 7.30-7.76 ppm.

FIG. 3 shows radiochemical purity analysis of the crude labeled mixture of ¹⁸⁸Re-DTPA-PEG-b-PCL using ITLC-SG. (R_{f} ¹⁸⁸Re DTPA-PEG-b-PCL=0.0; R_{f} ¹⁸⁸Re-DTPA=1.0).

FIG. 4 shows characterization of one embodiment of the nanoparticle of the present invention. (A) IR-780 micelles were imaged by TEM, and the scale bar is 200 nm (B) Size distribution of IR-780 micelles at a D/P ratio of 1:10 was analyzed by DLS. (C) Absorbance spectra were measured for empty micelles, free IR-780 iodide, and IR-780 micelles in PBS. (D) Temperature of IR-780-loaded micelles during 1.8 W/cm² NIR laser irradiation was profiled, with the data presented as mean±SD.

FIG. 5 shows radiochemical purity analysis of the ¹⁸⁸Re-DTPA-micelles using ITLC-SG.

FIG. 6 shows in (A) photothermal ablation and live/dead staining illustrated for HCT-116 cells that were treated with 0.6 W/cm² NIR irradiation (the treated region labeled as “laser”) for 10 min mediated by 10 μg/mL IR-780 micelles. The live cells are stained green with calcein-AM, and dead cells are stained red with PI. FIG. 6 also shows the cytotoxicities of IR-780 micelles (B) and free IR-780 iodide (C) in HCT-116 cells without or with 0.6 W/cm² NIR irradiation for 10 or 20 min.

FIG. 7 shows MicroSPECT/CT images and biodistribution of ¹⁸⁸Re-labeled IR-780 micelles in tumor mice bearing HCT-116. (A) ¹⁸⁸Re-labeled IR-780 micelles were injected, and then microSPECT/CT images were acquired 1, 4, and 24 h later. (B) ¹⁸⁸Re-labeled IR-780 micelles were intravenously injected into mice bearing HCT-116 tumors, and their biodistribution was determined 1, 4, 24, 48, and 72 h later. Each column represents the mean±SD.

FIG. 8 shows one embodiment of the present invention as follows: (A) time-lapse near-IR fluorescence (NIRF) imaged mice bearing HCT-116 tumors after intravenous injections of IR-780 micelles; (B) NIR fluorescence intensities and contrast index (CI) values quantified at the indicated time points in the tumor and normal regions, using the maximal NIRF signals in the nontumor regions; (C) near-IR fluorescence (NIRF) images; and (D) quantification of various organs at 24 h after intravenous injection of IR-780 micelles. Each column represents the mean±SD. The abbreviations indicate: H, heart; Li, liver; Sp, spleen; Lu, lung; K, kidney; and In, intestine.

FIG. 9 shows one embodiment of the present invention as follows: (A) schematic diagram illustrating the photothermal therapy of IR-780 micelles following NIR light irradiation; (B) intratumoral temperature profile during IR-780 micelle-mediated photothermal therapy measured as a function of time with thermocouple needles inserted in the center of the tumor while the tumor region was irradiated by the 1.8 W/cm² NIR laser for 5 min; (C) infrared thermographic map of the HCT-116 tumor treated with IR-780 micelles measured with a thermal camera after NIR irradiation; (D) temperature along the scan line in the corresponding thermal images in panel C quantified, with the shaded region corresponding to the tumor region exposed to NIR light.

FIG. 10 shows measured effects of PTT mediated by IR-780 micelles in mice bearing HCT-116 tumor. (A) Tumor volumes and (B) body weights were measured during the 27 day evaluation period in mice treated with PBS (control), NIR irradiation alone, IR-780 micelles alone, or IR-780 micelles plus NIR irradiation. Data indicate means and standard errors. (C) Representative mice treated with NIR irradiation alone or with IR-780 micelles equivalent to 1.25 mg/kg and 1.8 w/cm² NIR irradiation for 5 min were photographed over days 2-24. The red and black arrows indicate the NIR irradiation site and no NIR irradiation, respectively.

FIG. 11 shows histological and immunohistochemical analysis in HCT-116 xenograft tumors treated with IR-780 micelle-mediated photothermal therapy. (A) Tumor blocks were analyzed by hematoxylin and eosin (H&E) staining, NADPH-diaphorase staining (NADPH). More necrotic (N) tissue on the interior of the tumors was present when the tumors were treated with the combination of IR-780 micelles and NIR irradiation, which indicates the loss of NADPH-diaphorase activity. (B) Immunohistochemical staining of PCNA, TUNEL, HSP70, and HSP90 from the blue dotted squares in panel A. (C) Cellular proliferation was quantified by assessing the number of PCNA-positive cells per field at 200× magnification, and (D) apoptotic cells were quantified by the TUNEL method at 200× magnification. The results represent the mean±SD in 10 distinct regions from examining three tumors per group. The double star (**) indicates P<0.01.

FIG. 12 shows histopathological analysis in HCT-116 xenograft tumors treated with (A) PBS (Control), (B) NIR irradiation alone, or (C) IR-780 micelles+NIR irradiation. Tumor sections were analyzed by Hematoxylin & eosin staining (right) and NADPH-diaphorase staining (left). H&E staining of tumor treated with NIR laser irradiation alone shows tissue damage beneath the apical tissue surface, which was in agreement with NADPH-diaphorase staining. More necrotic (N) tissue (loss of NADPH-diaphorase activity) on the interior of the tumors was present when the tumors were treated with the combination of IR-780 micelles and NIR laser irradiation.

### DETAILED DESCRIPTION OF THE PREFERRED EMBODIMENTS

Various specific details are herein provided to provide a more thorough understanding of the invention.

### Materials

εe-Caprolactone, stannous octoate, and methoxy poly(ethylene glycol) (mPEG, MW=5000) were from Fluka (Milwaukee, Wis., USA), and fluorenylmethyloxycarbonyl-amino-poly(ethylene glycol) (Fmoc-NH-PEG-OH, Mₙ=5000 Da) was from Laysan Bio Inc. (Arab, Ala., USA). Before polymerization, mPEG was vacuum-dried at room temperature for 24 hours. All other HPLC grade solvents, including methanol, ethanol, n-hexane, dichloromethane (DCM), acetone, dimethyl sulfoxide (DMSO), acetonitrile, and tetrahydrofuran (THF) were from Tedia Inc. (Fairfield, Ohio, USA). Both DCM and THF were dried over calcium hydride (CaH₂) and distilled before use. Stannous (II) octoate (SnOct), 3-caprolactone (CL), 3-(4,5-dimethylthiazol-2-yl)-2,5-diphenyl tetrazolium bromide (MTT), diethylenetriaminepentaacetic acid dianhydride (DTPA dianhydride), calcein-AM, ropidium iodide (PI), and cyanine dye IR-780 iodine were from Sigma Aldrich (Milwaukee, Wis., USA).

Synthesis of mPEG-b-PCL and DTPA-PEG-b-PCL

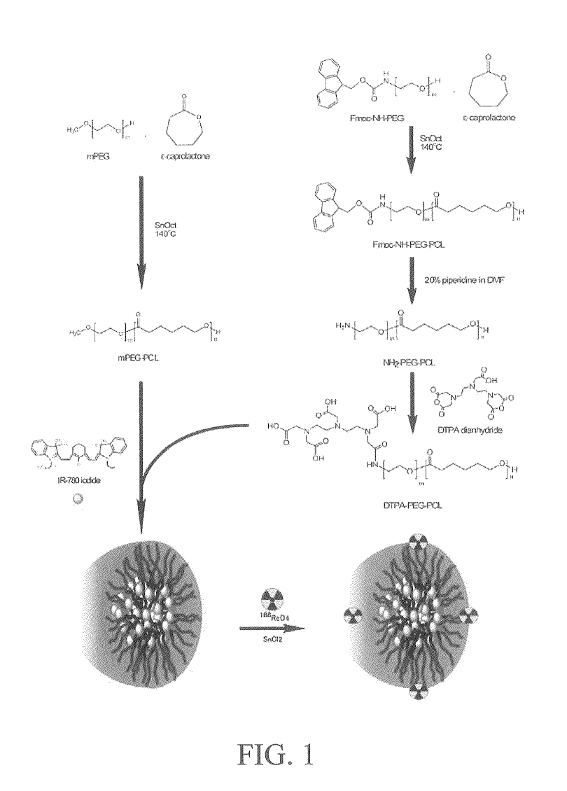

Methoxy poly-(ethylene glycol)-block-poly(ε-caprolactone) (mPEG-b-PCL) and fluorenylmethyloxycarbonyl-amino-poly(ethylene glycol)-block-poly(ε-caprolactone) (Fmoc-NH-PEG-b-PCL) amphiphilic block copolymers were synthesized by ring-opening polymerization of ε-caprolactone at 140° C. overnight in the presence of mPEGOH (MW=5000) and Fmoc-NH-PEG-OH (MW=5000) as a macroinitiator under stannous octoate (SnOct) catalysis (FIG. 1). The synthesized polymers were recovered by dissolving them in THF and then precipitating them in ice-cooled diethyl ether. The resultant precipitate was filtered and dried at room temperature under vacuum. The Fmoc-NH-PEG-b-PCL was deprotected by stirring Fmoc-NH-PEG-b-PCL in 2 mL of 20% piperidine in DMF for 2 h at room temperature. Then, the NH₂-PEG-b-PCL was purified by dialysis against water for 7 days, with the deionized water being changed twice per day. Finally, the NH₂-PEG-b-PCL residue as isolated as a sponge by lyophilization and kept for further use. The DTPA-PEG-b-PCL was prepared by conjugating DTPA dianhydride with the amino group of N₂HPEG-b-PCL. Briefly, the N₂H-PEG-b-PCL (100 mg, 7.1 μmol) was dissolved in 5 ml DMF in the presence of triethylamine (2.0 mg, 20 μmol). Then, DTPA dianhydride (7.1 mg, 20 μmol) dissolved in 1 ml DMF was added, and the mixture was stirred at room temperature for 24 hours. The product was collected by precipitation in diethyl ester and then filtrated and re-dissolved in THF. Finally, the mixture was transferred into dialysis bags (M_{w} cut-off 8000 Da; Spectrapor, Spectrum Laboratories Inc., San Diego, Calif.), and immersed in deionized water to remove any free DTPA. The DTPA-PEG-b-PCL residue was lyophilized prior to storage at 4° C. The molecular weights of the synthesized polymers were characterized by ¹H NMR (Bruker Avance 500 MHz FT-NMR) using deuterated chloroform (CDCl₃) as the solvent and gel permeation chromatography (GPC) using Waters 510 pump equipped with a Waters 410 differential refractometer. Tetrahydrofuran (THF) was used as the eluent at a flow rate of 1.0 mL/min Calibration used monodispersed polystyrene standards. The DTPA conjugation efficiency was evaluated by the radiolabeling yields of ¹⁸⁸Re-DTPAPEG-b-PCL, as analyzed by instant thin layer chromatography (ITLC), and by calculating the relative amounts of ¹⁸⁸Re-DTPAPEG-b-PCL and free ¹⁸⁸Re-DTPA (R_{f} ¹⁸⁸Re-DTPA-PEG-b-PCL=0; R_{f} ¹⁸⁸Re-DTPA=1).

PEG-b-PCL and Fmoc-NH-PEG-b-PCL were synthesized by a ring-opening polymerization of ε-caprolactone in the presence of either mPEG-OH or Fmoc-NH-PEG-OH, respectively (FIG. 1). Both mPEG-b-PCL and Fmoc-NH-PEG-b-PCL were characterized by ¹H NMR spectrum, and the molecular weights and polydispersity of copolymers were determined by GPC. The characteristics of mPEG₅ₖ-PCL₁₀ₖ, Fmoc-NH-PEG₅ₖ-PCL₁₀ₖ, and DTPA-PEG₅ₖ-PCL₁₀ₖ are summarized in Table 1.

TABLE 1
Characteristics of mPEG-b-PCL and DTPA-PEG-b-PCL Copolymers

| | | | | M_{w}/ | CMC^{d} | size^{e} |
|---|---|---|---|---|---|---|
| Sample | M_{n, Theo}^{a} | M_{n, NMR}^{b} | M_{n, GPC}^{c} | Mₙ^{c} | (wt %) | (nm) |
| mPEG₅ₖ-b-PCL₁₀ₖ | 15000 | 15400 | 20900 | 1.31 | 0.006 | 74 ± 32 |
| Fmoc-NH- | 15000 | 14200 | 15900 | 1.28 | | |
| PEG₅ₖ-b-PCL₁₀ₖ | | | | | | |
| DTPA- | | 14000 | 15100 | 1.42 | | |
| PEG₅ₖ-b-PCL₁₀ₖ | | | | | | |

^{a}Theoretical molecular weight based on feed ratio.
^{b}Calculated from 1H NMR data.
^{c}Determined by GPC.
^{d}CMC indicates critical micelle concentration.
^{e}As determined by DLS.

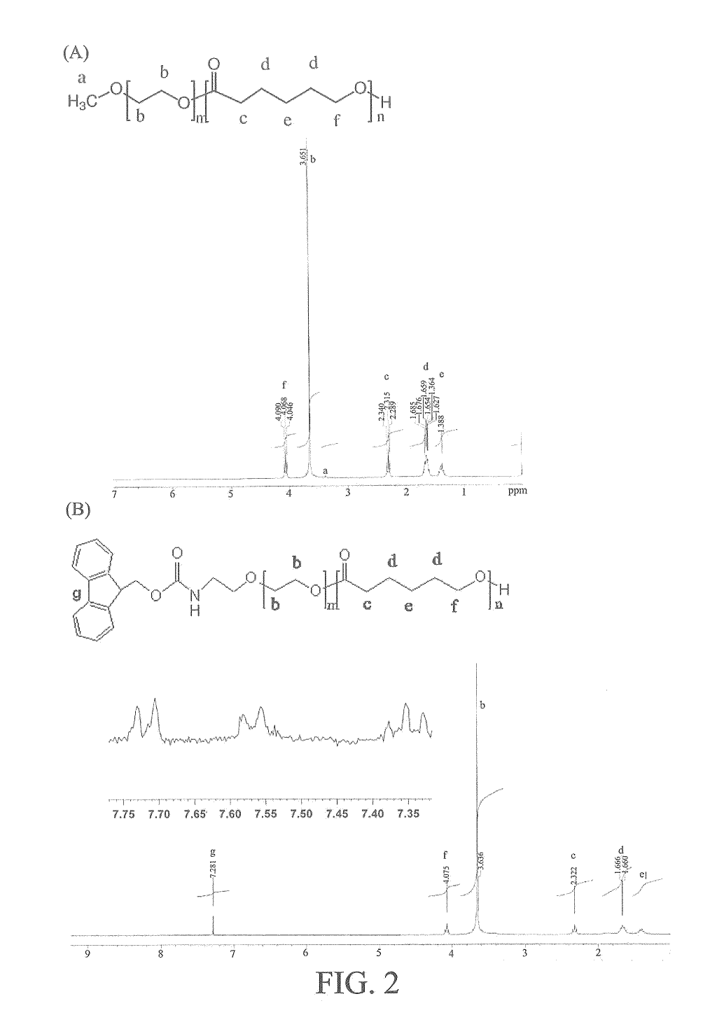

The characteristic resonances of both PCL (δM^{e}=1.37 ppm, δH^{d}=1.65 ppm, δH^{c}=2.28 ppm, δH^{f}=4.07 ppm) and mPEG (δH^{a}=3.39 ppm and δH^{b}=3.65 ppm) were observed, suggesting the coexistence of two blocks. The molecular weight (M_{n,NMR}) of PCL was determined by comparing the peak intensities of the methylene protons of the oxyethylene units (δM^{b}) of mPEG to the methylene protons (δH^{d}) of PCL (FIG. 2). This molecular weight (MW) was in good agreement with the theoretical MW that was calculated based on the feed ratio of s-CL to mPEG or Fmoc-NH-PEG.

The ¹H NMR spectrum of Fmoc-NH-PEG-b-PCL exhibited distinct resonance signals of Fmoc moieties at 7.30-7.76 ppm, which were not present in the spectrum of mPEG-b-PCL (FIG. 2). Analysis by GPC revealed a shift to earlier elution times for Fmoc-NH-PEG-b-PCL, relative to Fmoc-NHPEG-OH, which is consistent with an increase in MW distribution and indicates a successful ring-opening polymerization of ε-CL. The Fmoc-NH-PEG-b-PCL copolymer had a slight broadening of the GPC peaks and polydispersity compared to the Fmoc-NH-PEG macroinitiator.

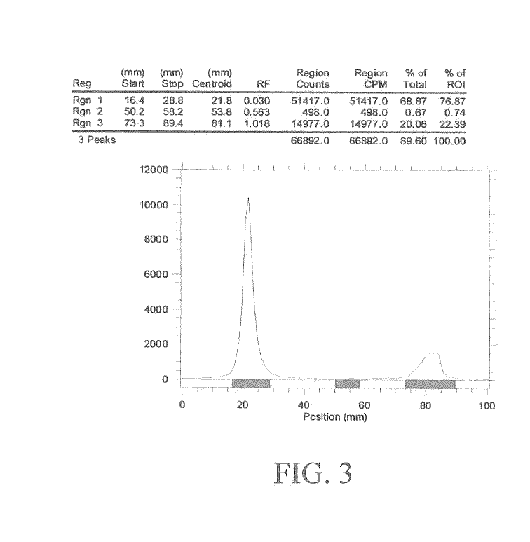

Amino-terminated PEG-b-PCL (H₂N-PEG-b-PCL) copolymers were prepared via deprotection of the Fmoc-NH-PEG-b-PCL that was accomplished by stifling Fmoc-NH-PEG-b-PCL with 20% piperidine in DMF. The NH₂-PEG-b-PCL copolymers were then purified by dialysis before being lyophilized to dryness. The DTPA-PEG-b-PCL was prepared by conjugating the DTPA dianhydride with the amino group of N₂H-PEG-b-PCL. The conjugation efficiency of DTPA dianhydride to NH₂-PEG-b-PCL was evaluated by ITLC that analyzed the efficiency of the ¹⁸⁸Re labeling of DTPA-PEG-b-PCL. It revealed that 76.8% of the radioactivity remaining at the origin corresponded to ¹⁸⁸Re-DTPA-PEG-b-PCL (FIG. 3). The copolymer MW of DTPA-PEG-b-PCL was determined to be about 14,000 Da by ¹H NMR spectroscopy and about 15,100 Da by GPC (Table 1).

### Preparing IR-780 Micelles and IR-780/DTPA Micelles

IR-780 micelles and IR-780-loaded/DTPA micelles (IR-780/DTPA micelles) were prepared by the cosolvent evaporation method. Briefly, a mixture of 10-40 mg of mPEG-b-PCL was dissolved in acetone with 2 mg of IR-780 iodide dye (D/P=1/5-1/20), or 36 mg of mPEG-b-PCL and 4 mg of DTPA-PEG-b-PCL in a ratio of 9:1 were dissolved in acetone with 2 mg of IR-780 dye (D/P was 1:20). These mixtures were added to saline while stirring with a rotor-stator device (Variomag Poly 15, H+P Labortechnik GmbH, Munich, Germany) at a speed of 550 rpm. The organic solvent was evaporated, while the solution was stirred overnight. Then, the solution was filtered through a 0.45 μm sterile filter (Millex GS, Millipore, Bedford, Mass., USA) to remove non-incorporated drug crystals and copolymer aggregates. The IR-780 micelles were lyophilized and then dissolved with DMSO. The concentration of IR-780 iodide was determined with a spectrophotometer using a quartz cell with a 1 cm path length at 786 nm. The drug encapsulation efficiency is the amount of drug encapsulated divided by the amount of drug added multiplied by 100%.

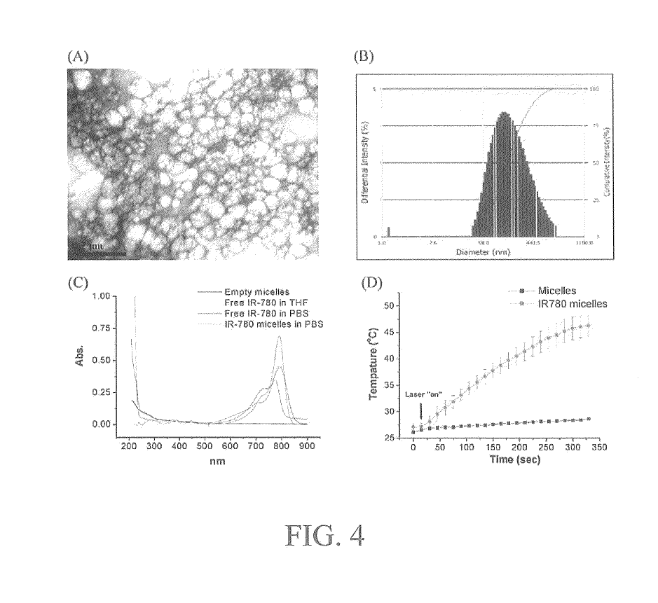

The IR-780-loaded micelles, as observed by TEM, had a spherical morphology with particle sizes in agreement with DLS (FIG. 4). The mPEG-b-PCL micelles had a size distribution about 100 nm in diameter. After IR-780 was loaded into micelles using various D/P ratios, DLS determined that the micelles ranged from 155 to 203 nm in size with various polydispersity indices (Table 2). Those IR-780-loaded micelles with a D/P ratio of 1:20 were employed, which had an encapsulation efficiency of 93.8%, and each micelle contained approximately 6414±641 IR-780 iodide dye molecules.

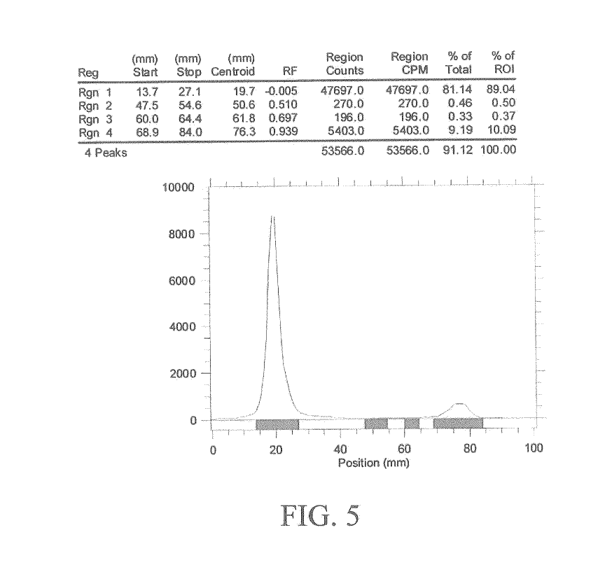

For example, the number of IR-780 iodide dye loaded into each micelle (Ndye) was calculated using the equation Ndye=Wdye/Mn, in which Wdye is the weight of IR-780 iodide dye loading per micelles and Mn is the molar mass of the IR-780 iodide dye (Mn=667). The IR-780 iodide dye loaded micelles with a D:P ratio of 1:20 were employed, which had an encapsulation efficiency of 93.8%. The weight average molecular weight of these micelles (Mw, micelle), obtained from static light scattering (SLS) using a Zetasizer Nano ZS90 apparatus (Malvern Instruments, Worcestershire, UK), was (77.0±7.7)×10⁶ g/mol as shown in FIG. 5. Further, the weight of IR-780 iodide dye loading per micelles (Wdye) was calculated using the equation: Wdye=Mw,micelle×feed weight ratio×encapsulation efficiency=[(77.0±7.7)×10⁶]×5%×93.8%≈(3.611±0.361)×10⁶ (g/mol). And the number of IR-780 iodide dye loaded into each micelle (Ndye) was calculated using the equation: Ndye=Wdye/Mn=[(3.611±0.361)×10⁶]/667=6414±641. Hence, each micelle contained approximately 6414±641 IR-780 iodide dye molecules.

The micelles with a D/P ratio of 1:5 or 1:10 had larger particle sizes and lower encapsulation efficiencies than those with a D/P ratio of 1:20 (Table 2). Drug encapsulation efficiency is a crucial factor in developing micelles or other drug delivery vesicles. Moreover, since a drug solution will be distributed all over the body, the EPR effect may preferentially distribute nanoparticles of 100-300 nm to the tumor, while the reticuloendothelial system will readily scavenge drug carriers with a diameter larger than 200 nm. The IR-780 micelle with a D/P ratio of 1:20, which exhibited efficient drug encapsulation and an ideal size suitable for future medical applications, was chosen as the drug carrier for further study.

TABLE 2
Characteristics of IR-780 Micelles

| | | encapsulation | drug content | mean size/nm |
|---|---|---|---|---|
| polymer | D/P ratio^{a} | efficiency (%)^{b} | (%)^{c} | (PDI)^{d} |
| m52 | 1:5 | 30.3 | 5.71 | 203.6 (0.436) |
| | 1:10 | 62.9 | 5.92 | 187.9 (0.317) |
| | 1:20 | 74.8 | 3.61 | 143.8 (0.236) |
| m510 | 1:5 | 34.6 | 6.47 | 172.2 (0.367) |
| | 1:10 | 63.3 | 5.95 | 165.7 (0.307) |
| | 1:20 | 93.8 | 4.47 | 155.0 (0.293) |

^{a}D/P ratio = weight of IR-780 iodide/weight of polymer.
^{b}IR-780 iodide encapsulation efficiency (%) = (weight of IR-780 iodide in the micelles/weight of the feeding IR-780 iodide) × 100%.
^{c}IR-780 iodide drug content (%) = (weight of IR-780 iodide)/(weight of IR-780 iodide t weight of polymer) × 100%.
^{d}As determined by DLS.

The IR-780 cyanine dye diluted in THF and IR-780 micelles in PBS strongly absorbed in the NIR region with a maximum wavelength (λₘₐₓ) at about 795 nm (FIG. 4C). Since IR-780 cyanine dye is lipophobic, it aggregates in aqueous buffer. The aggregation of lipophilic IR-780 iodide results in a broad and blueshifted absorption peak at λₘₐₓ=775 nm (as shown in FIG. 4C), which decreased the absorption from laser diode with a wavelength of 808 nm, resulting in reduced efficiency of PTT. In contrast, the IR-780-loaded micelles still exhibited a relatively strong absorbance in the NIR range in aqueous buffer, indicating that loading the lipophobic IR-780 cyanine dye in the micelles to encapsulate it did not change its photophysical properties. The temperature of the IR-780-loaded micelle medium increased rapidly during NIR irradiation and reached maximal temperature of approximately 46° C. after 5 min, while the empty micelles increased by 2.5° C. during NIR irradiation (FIG. 4D). These results indicate that most of the heat during NIR irradiation came from the IR-780 dye.

### Preparing ¹⁸⁸Re-Labeled IR-780 Micelles

The ¹⁸⁸Re with DTPA micelles were labeled by reacting a mixture of 1 mL of DTPA micelles, 100 μL of ¹⁸⁸Re-perrhenate (¹⁸⁸ReO₄, about 37 MBq), and 5 mg of stannous chloride for 2 h at 37° C. The radiolabeling yields of ¹⁸⁸Re-DTPA micelles were determined by ITLC using silica gel as the stationary phase and normal saline as the mobile phase. The chromatograms were analyzed by a radio thin layer chromatography imaging scanner (AR2000, Bioscan, Washington, D.C., USA).

The IR-780 iodide-loaded DTPA micelles (IR-780/DTPA micelles) were labeled with ¹⁸⁸Re by reacting IR-780/DTPA micelles, ¹⁸⁸Re-perrhenate, and stannous chloride for 2 h at 37° C. The ¹⁸⁸Re-labeled IR-780/DTPA micelles had high radioactivity and radiochemical purity (about 90%) as analyzed by ITLC (FIG. 5).

### Characterizing IR-780 Micelles

The mean diameter and polydispersity index (PDI) of the micelles were characterized with a Delsa Nano Particle Analyzer (Beckman Coulter, Fullerton, Calif.). The morphology of the micelles was observed by H-7650 transmission electron microscopy (TEM, Hitachi Ltd., Tokyo, Japan). The absorptions of the IR-780 iodide dissolved in 0.15 M NaCl buffer and of IR-780 micelles dispersed in phosphate buffer saline (PBS) were measured on a UV-vis spectrophotometer (BioMate 3S, Thermo Electron Corporation, Hudson, N.H., USA) with a quartz thermostatted cell with a 1 cm path length. The temperature profile of the IR-780 micelles during NIR irradiation was analyzed in a 24-well plate with a thermocouple needle. A total of 1 mL of about 100 μg/mL IR-780 micelles was added to one of the wells, the well was irradiated by the NIR laser at 1.8 W/cm², and the temperature of the well was measured continuously over 5 min

### In Vitro Cytotoxicity

The HCT116 human colon cancer cells were maintained in a humidified 5% CO₂ incubator at 37° C. in DMEM (Gibco BRL, Gaithersburg, Md., USA) supplemented with 10% heat-activated fetal bovine serum (FBS) and 1% antibiotics (antibiotic-antimycotic; Gibco). The HCT-116 cells were seeded onto 6-well plates at a density of 1×10⁶ cells per well and cultured.

The HCT-116 cells were incubated in media containing different concentrations of IR-780 micelles for 3 h and washed with PBS. Next, the cells were treated for 10 min with a laser diode with a wavelength of 808 nm at a power density of 0.6 W/cm². After the irradiation, the cells were stained for 30 min with 2 μM calcein-AM and 2 μM propidium idodide (PI) prior to imaging. Cell viability was visually determined with an X51 Olympus fluorescence microscope (Olympus Optical Co., Tokyo, Japan).

The cytotoxicity of treating HCT-116 cells with IR-780 micelles and NIR irradiation was additionally determined The HCT-116 cells were first seeded onto 96-well plates at a density of 10,000 cells per well and cultured. After 24 h, the cells were incubated in media with different concentrations of IR-780 micelles for 3 h and then washed with PBS. Next, the cells were treated with a laser diode with a wavelength of 808 nm at a power density of 0.6 W/cm² for 10 or 20 min. Cell viability was determined with the MTT assay and a scanning multiwell ELISA reader (Microplate Autoreader EL311, Bio-Tek Instruments Inc., Winooski, Vt., USA). The fraction of live cells was calculated by dividing the mean optical density obtained from treated cells by the mean optical density from untreated control cells.

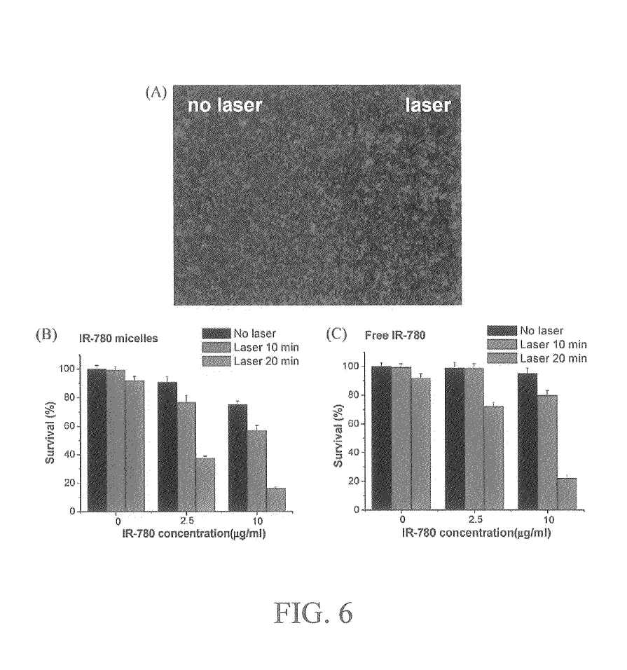

HCT-116 cells were used to evaluate the cytotoxicity of HCT-116 treated with IR-780 micelles plus NIR irradiation. The cells were treated with IR-780 micelles and NIR irradiation, and then live cells were stained with calcein AM, a nonfluorescent cell-permeating compound that is hydrolyzed by intracellular esterases in live cells into intensely fluorescent calcein, and dead cells with PI (FIG. 6A). Live cells were determined by the green fluorescence of calcein in the dark region. The light regions indicated cell death, where increased PI penetration and binding to nucleic acids produced a bright red fluorescence. The increased loss of cell viability in the irradiated regions confirmed that cell death was confined to the area treated by the IR-780 micelles with NIR irradiation. Exposing the cells to IR-780 micelles without NIR irradiation did not compromise cell viability.

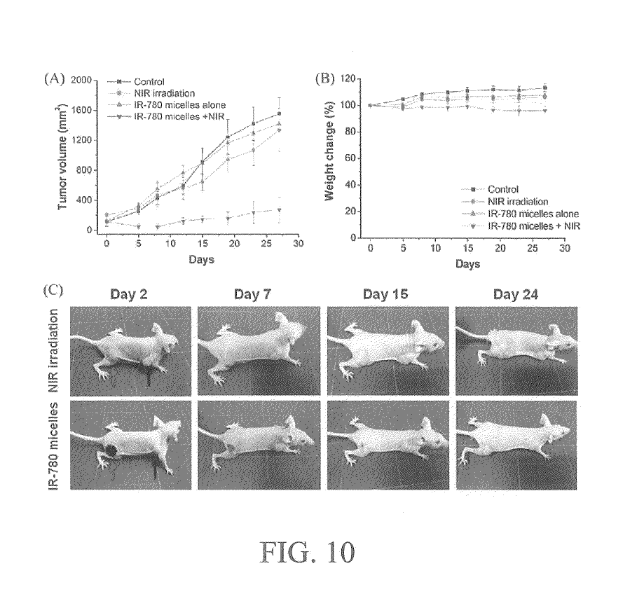

The cytotoxicity of IR-780 micelles and free IR-780 iodide in HCT-116 cells without or with NIR irradiation was also determined by the MTT assay. Treatment of the cells with only NIR irradiation for 10 or 20 min did not cause observation cell death (FIG. 6B). Treatment with IR-780 micelles without irradiation had more toxicity than free IR-780 iodide in HCT-116 cells. However, we observed no systemic toxicity of IR-780 micelles in nude mice, and this formulation also did not significantly affect body weights of the mice compared with control groups (as shown in FIG. 10B).

The HCT-116 cells treated with 2.5 μg/mL of IR-780 micelles and NIR irradiation (excess 14.4 and 53.5% of cells killed for 10 and 20 min of irradiation, respectively) significantly accelerates cell killing than that treated with 2.5 μg/mL of free IR-780 iodide and NIR irradiation (excess 0.3 and 26.8% of cells killed for 10 and 20 min of irradiation, respectively). The observation may be due to the aggregation of lipophilic IR-780 iodide in the aqueous medium, which reduces their photocytotoxicity and cellular uptake. The aggregation of lipophilic IR-780 iodide shows a broad and blue-shifted absorbance spectrum with a peak at λhd max=775 nm (as shown in FIG. 4C), which decreased the absorbance for laser diode with a wavelength of 808 nm, resulting in reduced efficiency of PTT. When HCT-116 cells were treated with high concentrations and NIR irradiation, it showed significant phototoxicity by IR-780 micelles (85% of cells killed after 20 min of irradiation) compared with that by free IR-780 iodide. These results indicate that IR-780 micelles can be activated by 808 nm laser diode and act as a potential formulation for PTT.

### Biodistribution of ¹⁸⁸Re-Labeled IR-780 Micelles and IR-780 Iodide by Micelle Formulas

After PTT mediated by the IR-780 micelles plus NIR irradiation, the cells were incubated in media for three hours, and then stained for 30 minutes with 2 μM calcein-AM and 2 μM propidium idodide (PI) prior to imaging. The calcein-AM (excitation at 495 nm and emission at 515 nm) stained live cells green, and the PI (excitation at 535 nm and emission at 617 nm) stained dead cells red. Cell viability was visually determined with an X51 Olympus fluorescence microscope (Olympus Optical Co., Tokyo, Japan).

The images were acquired with a microSPECT/CT scanner system (XSPECT, Gamma Medica, Northridge, Calif., USA). The SPECT images used a low-energy, high-resolution collimator and were taken 1, 4, and 24 hours after the micelles were intravenously injected. During the imaging, the mice were kept still by inhaling anesthetic isoflurane (ABBOTT, Kent, England). The SPECT imaging was followed by acquiring CT images using a 50 kV, 0.4 mA X-ray source with 256 projections while the animal was in the exact same position. The CT images were reconstructed with COBRA_Exxim software (Exxim Computing Corporation, Pleasanton, Calif., USA) and the SPECT images with LumaGEM software (Segami, Columbia, Md., USA). The SPECT/CT images were fused with IDL 6.0 software (RSI Inc, Boulder, Colo., USA).

Female BALB/c athymic (nut/nut) mice that were 5-6 weeks old were purchased from the National Laboratory Animal Center (Taipei, Taiwan). Tumors were initially established by subcutaneously injecting a mixture of 1×10⁶ HCT-116 cells, matrigel, and DMEM. Tumor sizes and body weights were measured every 3 days for the duration of the experiment. Tumor volume was calculated as π/6ab², where “a” is the length and “b” is the width of the tumor.

Mice received an intravenous injection of ¹⁸⁸Re-labeled IR-780 micelles, equivalent to 22 MBq of ¹⁸⁸Re, when the tumors reached a volume of 150 to 200 mm³ The distribution of ¹⁸⁸Relabeled IR-780 micelles in the mice bearing HCT-116 tumors was evaluated by microSPECT/CT images at 1, 4, and 24 h after the micelles were intravenously injected.

The mice were sacrificed by cervical vertebra dislocation at 24 and 96 h after the intravenous administration of ¹⁸⁸Relabeled IR-780 micelles. The plasma, tumor, and normal tissue were collected, and the uptake of radioactivity was measured by a γ counter. The distribution data were expressed as the percentage of injected dose (ID). The biodistribution of IR-780 iodide was studied by injecting 1.25 mg/kg IR-780 micelles intravenously through a tail vein of mice bearing HCT-116 tumors and was imaged 1, 4, 24, 48, and 96 h after the injection with an IVIS imaging system (Xenogen, Alameda, Calif., USA). The mice were anesthetized with a mixture of oxygen and isoflurane, and were placed on a 37° C. animal plate. The near-infrared fluorescence (NIRF) data were collected with a two second exposure time and an ICG filter set with excitation at 710-760 nm and emission at 810-875 nm. All data were calculated using the region-of interest (ROI) function of the Living Image® software (Caliper Life Sciences Inc, Hopkinton, Mass., USA). Dye accumulation and retention in tumors was evaluated by calculating the contrast index (CI) values. The CI was measured according to the formula CI=(Ftumor−Fauto)/(Fnorm−Fauto). The Ftumor value is the fluorescence mean intensity of the tumor region, and the Fnorm value is that of the normal region. The Fauto value is the autofluorescence from the corresponding region measured before injection. The tumor-bearing mice were sacrificed 48 h after the IR-780 micelles were injected, and then the tumor, heart, liver, spleen, lung, kidneys, and intestine were harvested for isolated organ imaging to estimate the tissue distribution of IR-780 micelles.

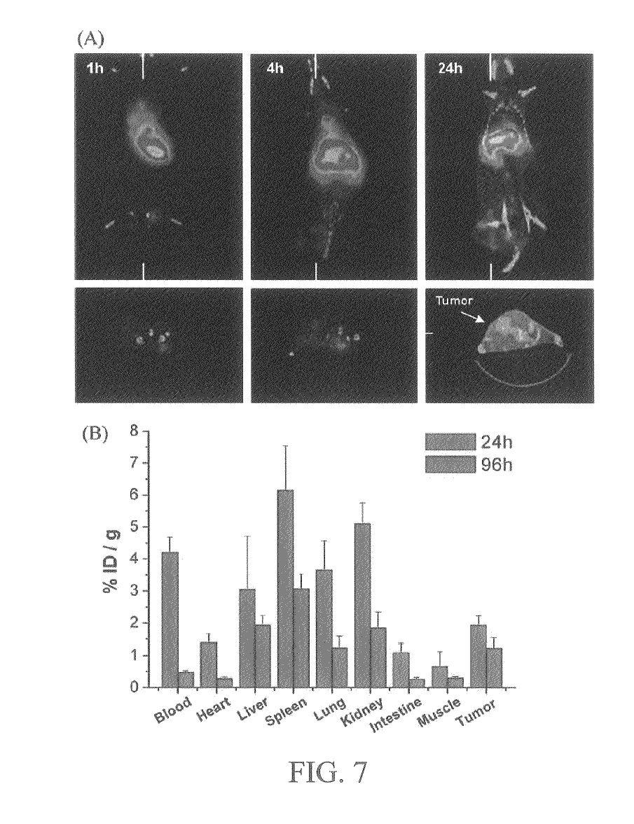

The biodistribution of ¹⁸⁸Re-labeled IR-780 micelles was evaluated in tumor and normal tissues of mice bearing HCT-116 human colon cancer xenografts. Images obtained by microSPECT/CT revealed that radioactivity accumulated in the spleen, liver, and tumor at 24 h after the injection of ¹⁸⁸Re-labeled IR-780 micelles, and that the tumors were targeted by the radioactivity (FIG. 7A). Biodistribution of ¹⁸⁸Re-labeled IR-780 micelles was also performed by γ-counting. The results indicated that the ¹⁸⁸Re-labeled micelles were widely and rapidly distributed into most tissues and the tumors, with the highest accumulations occurring in the spleen, followed by liver, kidney, lung, and tumor at 24 h after injecting micelles (FIG. 7B). After 96 h, the accumulation of radioactivity in all tissues and in the tumor decreased, with the spleen still having the highest radioactivity. This high radioactivity may be due to filtering by the splenic capillary bed that removed some large particles or their aggregates. The percentage ID per gram of ¹⁸⁸Re-labeled micelles decreased slowly at the tumor site from 1.93±0.30% ID/g at 24 h after the injection to 1.23±0.31% ID/g at 96 h, and it decreased quickly in the blood and most tissues. The tumor to muscle ratio of ¹⁸⁸Re-labeled micelles increased from 1.91±1.71 at 24 h after the injection to 4.27±1.48 at 96 h, which corresponds well to the EPR effects of the nanoparticles. Thus, amphiphilic-block-copolymer-based micelles appear to be an ideal candidate carrier that can “passively” target tumors, which is an ability that may improve antitumor efficacy and reduce the toxicity to and nonspecific targeting of normal cells that accompanies most chemotherapy or PTT.

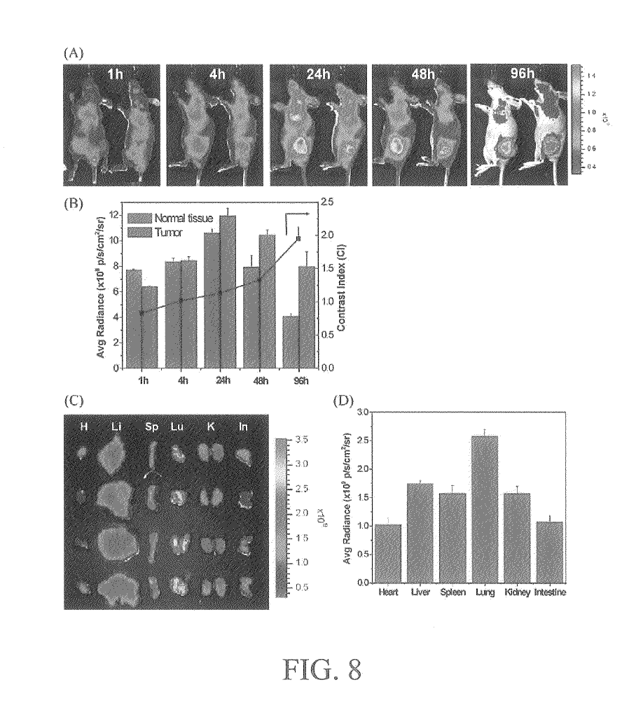

The in vivo real-time biodistribution of IR-780 iodide in HCT-116 tumor-bearing mice that were injected intravenously with ¹⁸⁸Re-labeled IR-780 micelles is characterized through NIR fluorescence imaging with an IVIS imaging system. The IR-780 iodide had a time-dependent biodistribution and tumor accumulation in mice bearing HCT-116 tumors (FIG. 8A). The whole bodies of the mice had clear NIRF signals during the first 24 h that decreased as time passed. The NIRF signals were visible in the tumor region for 96 h. The intensity of the NIRF signals in the tumor and normal chest regions were quantified and normal chest regions and the contrast index (CI) values at various time points after the IR-780 micelles were injected (FIG. 8B). The NIRF signal intensities of tumors gradually increased compared with the normal region after injections. The maximal NIRF signals in the non-tumor regions of whole body were selected to calculate the CI. The CI values increased from 1.01 to 1.95 over the time course of the IR-780 micelle injections (FIG. 8B), and the maximum CI values occurred 96 h after the injections, which is a result that favors the reduced skin phototoxicity and enhanced antitumor efficacy of cyanine-based PTT. The heart, liver, spleen, lung, kidneys, and intestine were isolated to evaluate the tissue distribution of IR-780 micelles by NIRF imaging 24 h after the IR-780 micelles were injected (FIG. 8C), and their signals were quantified (FIG. 8D). Because the lungs had higher concentrations of IR-780 iodide, the NIRF signals from the chest of mice were clearly visualized by whole body imaging during the experiment period (FIG. 8A). Comparing the biodistribution of radioactivity from the ¹⁸⁸Re-labeled IR-780 micelles, which represent the biodistribution of the nanocarrier, the highest concentration of IR-780 iodide was detected in the lungs. This may result from filtering by the tissue capillary bed that ruptured the structure of the micelle and caused the drug to be released and redistributed to other organs.

### Temperature Measurements

The intratumoral temperature increases upon NIR irradiation were determined by injecting 1.25 mg/kg IR-780 micelles through a tail vein into mice bearing HCT-116 tumors. Control mice were injected with 100 μL of empty micelles (equivalent to 25 mg/kg). The temperatures of the tumor tissues during NIR irradiation were measured 96 h after the injections with thermocouple needles (127 μm diameter, T-type, copper-constantan thermocouple, Omega Engineering, Stamford, Conn.) connected to a data acquisition system (TC-2190, National Instruments, Austin, Tex.). First, the 23 gauge needles intratumorally injected into the center of tumor about 3-4 mm in depth. Next, the thermocouples were inserted into the tumor through the 23 gauge needles, while the tumor region was exposed to 1.8 W/cm² NIR light for 5 min with a laser diode (λ=808 nm). All data were analyzed with Matlab (Mathworks, Natick, Mass., USA). The distribution of tumoral temperature after NIR irradiation was examined with an IR thermographic camera (F30s, NEC Avio Infrared Technologies Co., Ltd., Tokyo, Japan) in the mice treated with the IR-780 micelles.

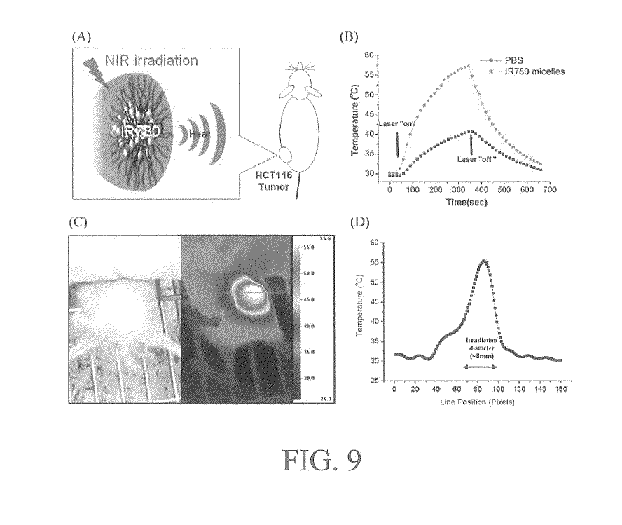

The intratumoral temperature profiles were measured during PTT mediated by IR-780 micelles (FIG. 9). Thermocouple needles were inserted in the center of tumor as a function of time, while the tumor region was irradiated by a 1.8 W/cm² NIR laser for 5 min. After the 5 min of NIR irradiation, the tumors treated with IR-780 micelles had a temperature increase of about 27° C., which exceeds the damage threshold needed to induce irreversible tissue damage. In contrast, the PBS-treated tumor for the same NIR irradiation resulted in a temperature increase of about 10° C. (FIG. 9B), which is insufficient to irreversibly damage tissue.

The spatial distribution of temperatures in the tumors of mice treated with PTT mediated by IR-780 micelles was observed with a thermal imaging camera (Thermo Shot F30, NEC Avio Infrared Technologies Co., Ltd.) (FIG. 9C). Excluding the region exposed to NIR irradiation, the maximum body temperature was about 36° C., corresponding to the normal body temperature of mice. For tumor regions treated with IR-780 micelles and exposed to NIR irradiation, the temperature along the scan line was quantitated, and the maximum tumor temperature increased to 56.6° C. (FIG. 9D), which was similar to the temperature measured by the thermocouple needle.

### Antitumor Efficacy of the IR-780 Micelles Upon NIR Irradiation

Treatments were started when the tumors reached a volume of 100 to 150 mm³. The mice were divided into groups of five mice each that were treated with the PBS control, the NIR irradiation alone, the IR-780 micelles, or the combination of IR-780 micelles and NIR irradiation. The IR-780 micelles were administered via tail vein injections at doses equivalent to 1.25 mg/kg of IR-780 iodide, and 96 h after the micelles were administered was designated as day 0. On day 0, the tumors were exposed to the NIR laser with a spot size of 5 mm at 1.8 W/cm² for 5 min. The tumor size and change in body weight of each mouse were recorded. The percentage of tumor growth inhibition (TGI) was calculated from the relative tumor volume on day 27 and is presented as percent reduction in the mean tumor volume in experimental groups compared with saline-treated control groups.

It was investigated how effectively PTT using IR-780 micelles on HCT-116 tumors in nude mice reduced tumor growth in vivo (FIG. 10). Control tumors treated with PBS, only the NIR irradiation, or only IR-780 micelles grew rapidly and uniformly, with no statistically significant differences in final tumor sizes (P=0.24). This indicated that tumor growth was not affected by either IR-780 micelles or NIR irradiation alone. In contrast, when the tumor volume was measured 27 days after PTT mediated by IR-780 micelles, it was reduced (mean tumor volume 271±168 mm³) compared with control tumors (1556±216 mm³) and TGI was 82.6% (P<0.01).

### Necropsy and Immunohistochemical Analysis

After the mice were sacrificed, the tumors were excised and fixed in formalin and embedded in paraffin for immunohistochemical staining and for hematoxylin and eosin staining. The tumor blocks, which were paraffin-embedded and 5 mm thick, were analyzed by immunohistochemical staining for proliferating cell nuclear antigen (PCNA), heat shock protein 70 (HSP70), and heat shock protein 90 (HSP90). Edogenous peroxidase activity was quenched with 3% hydrogen peroxide for 15 minutes, and then tumor blocks were blocked with 10% normal goat serum for 15 minutes and rinsed three times with PBS for two minutes. Consecutive blocks were incubated overnight at 4° C. with antibodies specific for HSP70 (rabbit anti-human, diluted 1:50, Cell Signaling Technology Inc., Danvers, Mass., USA), HSP90 (rabbit anti-human, diluted 1:50, Cell Signaling), and PCNA (mouse anti-PCNA, clone PC 10, Sigma). The blocks were again rinsed with PBS, and then incubated at room temperature with biotinylated secondary antibodies for 30 minutes. Finally, an avidin-biotin complex was applied and visualized with 3, 30-diaminobenzidine tetrahydrochloride chromogen. The immunostaining was applied and visualized by using Histostain-Plus kits (Zymed Laboratories, Inc., San Francisco, Calif., USA). The terminal deoxynucleotidyl transferase dUTP nick end labeling (TUNEL) assay was carried out with the DeadEnd Colorimetric TUNEL System (Promega, Fitchburg, Wis., USA). The NADPH-diaphorase staining was carried out to demonstrate necrosis. Tissue viability was analyzed by reacting the samples for 20 min at room temperature with NADPH-diaphorase reaction solution (10 mL of 10 mmol/L phosphate buffered saline, pH 7.4, containing 10 mg NADPH, and 5 mg nitroblue tetrazolium).

Body weight loss was used as a measure of treatments-induced toxicity (FIG. 10B). The body weights of both control and treatment groups were monitored throughout the experimental period, and mice that lost over 20% of their original body weight were sacrificed. By day 27, the control groups treated with PBS or only the NIR irradiation gradually had increased their body weights by 6-11%, and those treated with the IR-780 micelles increased by 7%. These values were not significantly different between the control groups, which suggested that the dye dose was reasonably well-tolerated. It has been reported that heptamethine indocyanine dyes had no systemic toxicity in normal C-57BL/6 mice and did not affect body weights of the mice. No abnormal histopathology was seen in vital organs harvested from mice at the time of sacrifice. Intravenous injection with 100 nmol of IR-780 iodide, which was about 2.7 times higher than the dose we used in our in vivo studies, did not cause systemic toxicity. Mice treated with IR-780 micelles plus NIR irradiation lost 4% of their weight at day 27. This weight loss was not significantly different from the control groups, indicating that photothermal therapy mediated by IR-780 micelles did not result in unacceptable toxicity.

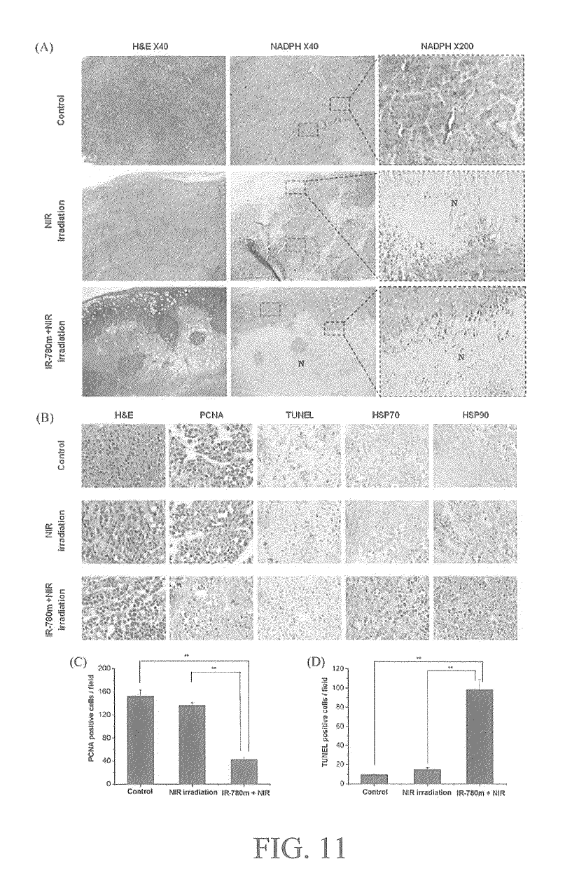

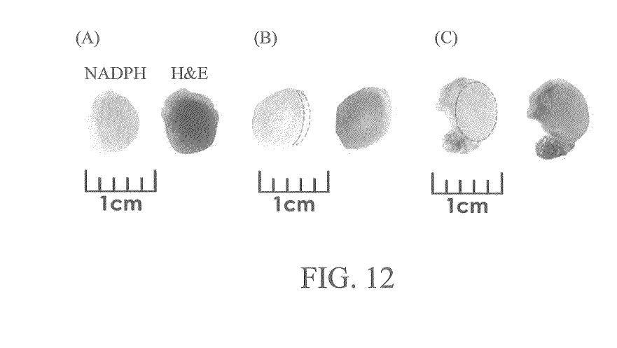

To further determine the effect of IR-780 micelle-mediated photothermal therapy in vivo, subcutaneously, tumors underwent immunohistochemical analysis (FIG. 11). Tumor tissues stained with hematoxylin and eosin had different tissue morphologies between treatment groups. As shown in FIG. 11A, common markers of thermal damage in tumors treated with PTT mediated by IR-780 micelles plus NIR irradiation, such as coagulation, vacuolation, and loss of nuclear staining, were identified. The blocks were stained with nicotinamide adenine dinucleotide phosphate (NADPH)-diaphorase staining for the assessment of tissue viability. Necrotic tissue shows loss of NADPH-diaphorase activity. The immunohistochemical analysis revealed that tumors treated with NIR irradiation alone had limited loss of NADPH-diaphorase activity at the surface of tumor, which was proximal to the incident laser (as shown in FIG. 8A and FIG. 12B). Maximal temperature changes were found to occur about 1 mm beneath the apical surface. This behavior may be the product of higher photon densities in this region, which is a phenomenon often seen in highly scattering mediums like tissue. In contrast, tumors treated with IR-780 micelle-mediated PTT had prominent necrosis and vacuolation. Necrotic features caused by the loss of NADPH-diaphorase activity were observed at the interior of the tumors. The maximum treatable depths of IR-780 micelle-mediated PTT appeared to be about 5-6 mm (FIG. 12C). These results indicate that NIR irradiation induced irreversible tissue damage mainly in the IR-780 micelle-treated tumor tissue.

Proliferating cell nuclear antigen (PCNA) immunolocalization can be used as an index of cell proliferation and may define the extent of departure from normal growth control. The PBS control tumors had a mean of 151.5±11.3 PCNA positive cells, and the tumors treated only with the NIR irradiation had a mean of 135.7±5.8 (FIG. 11B), which were not significantly between these two groups. The tumors treated with PTT mediated by IR-780 micelles plus NIR irradiation had decreased cell proliferation as detected by PCNA expression (mean±SD=48.4±4.5) in the viable, nonnecrotic regions (FIG. 11B). Their cell proliferation was significantly lower than those treated with only NIR irradiation or with PBS (both P<0.01), so combining NIR irradiation with IR-780 micelles reduced the number of proliferating cells within the subcutaneous tumors (FIGS. 8A-8B).

Apoptotic cells in each treatment were identified by the terminal deoxynucleotidyl transferase dUTP nick end labeling (TUNEL) method. TUNEL is a method for detecting DNA fragmentation, which results from apoptotic signaling cascades, by labeling the terminal end of nucleic acids. The PBS control group tumors had a mean of 9.3±2.6 apoptotic cells, and those tumors treated only with NIR irradiation had a mean of 14.6±3.2. The viable, non-necrotic regions in tumors treated with PTT mediated by IR-780 micelles plus NIR irradiation had more apoptotic tumor cells (mean±SD=98.2±10.8) than either control group (for both, P<0.01).

Since HSPs are induced by temperatures above 43° C., they serve as endogenous markers of thermal stress. Tumors treated only with PBS had minimal expression of HSPs, while those treated with only NIR irradiation had more HSP90 expression induced in the viable, non-necrotic regions of tumors, which were close to the incident laser (FIG. 11). Tumors treated with PTT mediated by IR-780 micelles plus NIR irradiation had enough temperature elevation to induce necrosis at the inner of tumor, which prevented the induction of HSPs, though the viable tumor surrounding the necrotic region did have induced HSPs. These results suggest that PTT mediated by IR-780 micelles plus NIR irradiation can extend the depth of thermal therapy of tumors, resulting in inner necrosis and peripheral expression of HSPs. Measuring HSPs can also demarcate thermally treated regions since HSPs allow cells to adapt to gradual changes in their environment and to survive conditions that would otherwise be lethal through suppressing apoptosis and enhancing resistance to therapies. Thus, measuring HSPs may aid in future searches for optimal conditions for PTT mediated by IR-780 micelles.

### Statistical Analysis

All data are expressed as mean±standard deviation. The significance of difference in this study between groups was analyzed by the t-test. A value of P<0.05 was considered statistically significant.

In one embodiment of the present invention, IR-780 iodide-loaded micelles, which both acted as NIR contrast agents for optical imaging and were labeled with the radionuclide rhenium-188 (¹⁸⁸Re) for nuclear imaging, have been prepared and characterized. It has been demonstrated that the NIR dye, IR-780 iodide, could serve as a photosensitizing agent for photothermal therapy of cancer since using IR-780 micelles to generate heat upon NIR irradiation resulted in thermal destruction of colon cancer both in vitro and in vivo. Measurements of the viable regions around necrotic regions of tumors found that these treatments decreased the cell proliferation as measured by PCNA expression, increased apoptotic cells as measured by TUNEL, and increased the expression of HSPs. These results indicate that irreversible tissue damage was induced by PTT mediated by the IR-780 micelles plus NIR irradiation in treated tumors. This platform permits image-guided drug delivery. The tumor accumulation, intratumoral distribution, and kinetics of the drug can be monitored in real-time. This platform allows diagnosis and therapeutics to be combined in optical/nuclear imaging and PTT. The ¹⁸⁸Re-labeled IR-780 micelles potentially offer multifunctional modalities for the near-infrared (NIR) fluorescence and nuclear imaging and for photothermal therapy of cancer.

In another embodiment of the present invention, multifunctional micelles for optical and nuclear imaging and for PTT were prepared. Two imageable components were incorporated into this micelle, a NIR dye and a radionuclide, which created a multifunctional drug delivery system that permitted image-guided drug delivery and real-time monitoring of the accumulation of the drug in the tumor, the intratumoral distribution, and the kinetics of drug release. It has been demonstrated that IR-780 iodide-loaded micelles (IR-780 micelles), which were labeled with the radionuclide rhenium-188 (¹⁸⁸Re), can combine the modalities of targeting, imaging, and drug delivery on one nanocarrier. This multifunctional micelle presents simultaneous optical and nuclear imaging and treatment capacities in one delivery system, using NIR fluorescence imaging, microSPECT/CT imaging, and photothermal cancer ablation. The size and morphology of IR-780 micelles were determined by dynamic light scattering (DLS) and transmission electron microscopy (TEM), and their encapsulation efficiency and optical properties were also analyzed. Cellular cytotoxicity by the IR-780 micelles upon NIR irradiation was evaluated in human colon cancer HCT-116 cells, and a xenograft model of these cells investigated the biodistribution, SPECT imaging, generation of heat, and photothermal cancer ablation of IR-780 micelles.

What is claimed is:

1. A nanoparticle for detecting or treating a tumor, comprising:
a plurality of polymer backbones, each including a hydrophobic region, a hydrophilic region, and a chelating region; and
at least one first detectable substance bound to the chelating region of the polymer backbone,
wherein the hydrophobic regions of the polymer backbones form a core block, and the hydrophilic regions of the polymer backbones form a shell block surrounding the core block.

2. The nanoparticle according to claim 1, wherein the first detectable substance is a radionuclide.

3. The nanoparticle according to claim 2, wherein the radionuclide is selected from the group consisting of Fluorine-18, Copper-64, Technetium-99m, Indium-111, Iodine-123, Iodine-131, Holmium-166, Rhenium-188, Gold-198, and a combination thereof.

4. The nanoparticle according to claim 3, wherein the radionuclide is Rhenium-188 or Iodine-131, and the tumor is selected from the group consisting of liver cancer, colon cancer, breast cancer, lung cancer, thyroid cancer, neuroblastoma, glioblastoma, lymphoma, myeloma, and a combination thereof.

5. The nanoparticle according to claim 1, wherein the tumor is selected from the group consisting of lymphoma, Hodgkin's disease, myeloid leukemia, bladder cancer, head and neck cancer, brain cancer, neuroblastoma, glioblastoma, kidney cancer, lung cancer, myeloma, ovarian cancer, cervical cancer, bone cancer, thyroid cancer, adrenal gland cancer, cholangiocarcinoma, pancreatic cancer, skin cancer, liver cancer, testicular cancer, melanoma, colon cancer and breast cancer.

6. The nanoparticle according to claim 1, further comprising a second detectable substance bound to the hydrophobic region or the hydrophilic region of the polymer backbone.

7. The nanoparticle according to claim 6, wherein the second detectable substance is a visible or near infrared detectable substance.

8. The nanoparticle according to claim 7, wherein the second detectable substance is selected from the group consisting of fluorescein, fluorescein isothiocyanate (FITC), rhodamine, Texas Red, cyanine dye, cy3, cy5, cy5.5, cy7, cy7.5, Alexa fluor dye, heptamethycyanine, indocyanine green (ICG), IR-780, IR-783, ADS7800H, NIR-797 isothiocynate, and a combination thereof.

9. The nanoparticle according to claim 1, wherein the hydrophilic region comprises at least one of polyethylene glycol and polypropylene glycol, and the hydrophobic region comprises at least one of polycaprolactone, polybutyrolactone and polyvalerolactone.

10. The nanoparticle according to claim 1, further comprising crosslinkages between the polymer backbones.

11. The nanoparticle according to claim 1, wherein the polymer backbones form a micelle.

12. The nanoparticle according to claim 1, further comprising an anti-cancer drug bound to the polymer backbone.

13. The nanoparticle according to claim 12, wherein the anti-cancer drug is selected from the group consisting of 7-ethyl-10-hydroxycamptothecin (SN-38), camptothecin (CPT), paclitaxel, doxorubin, 17-(Allylamino)-17-demethoxygeldanamycin (17-AAG), celecoxib, capecitabine, docetaxel, epothilone B, Erlotinib, Etoposide, GDC-0941, Gefitinib, Geldanamycin, Imatinib, Intedanib, lapatinib, Neratinib, NVP-AUY922, NVP-BEZ235, Panobinostat, Pazopanib, Ruxolitinib, Saracatinib, Selumetinib, Sorafenib, Sunitinib, Tandutinib, Temsirolimus, Tipifamib, Tivozanib, Topotecan, Tozasertib, Vandetanib, Vatalanib, Vemurafenib, Vinorelbine, Vismodegib, Vorinostat, ZSTK474 and a combination thereof.

14. A method for detecting or treating a tumor, comprising administering a nanoparticle to a subject in need thereof,
wherein the nanoparticle comprises a plurality of polymer backbones, each including a hydrophobic region, a hydrophilic region and a chelating region, and at least one first detectable substance bound to the chelating region of the polymer backbone, and
wherein the hydrophobic regions of the polymer backbones form a core block, and the hydrophilic regions of the polymer backbones form a shell block surrounding the core block.

15. The method according to claim 14, wherein the first detectable substance is a radionuclide selected from the group consisting of Fluorine-18, Copper-64, Technetium-99m, Indium-111, Iodine-123, Iodine-131, Holmium-166, Rhenium-188, Gold-198, and a combination thereof.

16. The method according to claim 14, wherein the nanoparticle further comprises a second detectable substance bound to the hydrophobic region or the hydrophilic region of the polymer backbone.

17. The method according to claim 15, wherein the second detectable substance is a visible or near infrared detectable substance selected from the group consisting of fluorescein, fluorescein isothiocyanate (FITC), rhodamine, Texas Red, cyanine dye, cy3, cy5, cy5.5, cy7, cy7.5, Alexa fluor dye, heptamethycyanine, indocyanine green (ICG), IR-780, IR-783, ADS7800H, NIR-797 isothiocynate, and a combination thereof.

18. The method according to claim 16, further comprising detecting the first or second detectable substance by single-photon emission computed tomography (SPECT), positron emission tomography (PET), a radiation image system or a fluorescent image system.

19. The method according to claim 14, wherein the tumor is selected from the group consisting of lymphoma, Hodgkin's disease, myeloid leukemia, bladder cancer, head and neck cancer, brain cancer, neuroblastoma, glioblastoma, kidney cancer, lung cancer, myeloma, ovarian cancer, cervical cancer, bone cancer, thyroid cancer, adrenal gland cancer, cholangiocarcinoma, pancreatic cancer, skin cancer, liver cancer, testicular cancer, melanoma, colon cancer and breast cancer.

20. A composition for detecting and treating a tumor, comprising the nanoparticle of claim 1 and a pharmaceutical acceptable excipient thereof.
